(19) United States
(12) Patent Application Publication
(10) Pub. No.: US 2026/0027017 A1
(43) Pub. Date: Jan. 29, 2026

(54) PERSONAL HYGIENE WIPES AND METHODS FOR MAKING AND USING THE SAME

(71) Applicant: William H. Cross, III, Waco, GA (US)

(72) Inventor: William H. Cross, III, Waco, GA (US)

(21) Appl. No.: 19/278,860

(22) Filed: Jul. 24, 2025

Related U.S. Application Data
(60) Provisional application No. 63/674,840, filed on Jul. 24, 2024.

(51) Int. Cl. A61K 8/02; A61K 8/49; A61K 8/63; A61K 8/9789; A61K 8/9794; A61Q 17/00

(52) U.S. Cl. CPC A61K 8/0208; A61K 8/494; A61K 8/4946; A61K 8/63; A61K 8/9789; A61K 8/9794; A61Q 17/00

(57) ABSTRACT

In accordance with the purpose(s) of the present disclosure, as embodied and broadly described herein, the disclosure, in one aspect, relates to personal hygiene wipes designed to address the unique skin challenges faced by individuals. In one aspect, the personal hygiene wipes described herein can provide relief to a subject that is wearing or using medical devices and topically worn appliances. In another aspect, the use of the wipes described herein can treat or prevent the onset of medical adhesive-related skin injury (MARSI), which is a persistent and debilitating injury for a wide range of patient groups.

### CROSS-REFERENCE TO RELATED APPLICATIONS

This application claims the benefit of and priority to U.S. Provisional Patent Application No. 63/674,840, filed on Jul. 24, 2024, the contents of which are incorporated by reference herein in their entireties.

### BACKGROUND

Personal hygiene wipes are widely used for skin cleansing. However, existing formulations often fall short in addressing the specific needs of individuals using medical devices (e.g., insulin pumps, continuous glucose monitors) or topically worn appliances. These individuals frequently experience skin irritation, and allergic reactions due to the repeated application and removal of adhesives.

One significant problem in the medical field is medical adhesive-related skin injury (MARSI). Medical adhesive-related skin injury (MARSI) is skin damage that can occur when medical adhesives, such as tapes, dressings, or patches, are not applied, selected, or removed properly. MARSI can cause lesions that remain for at least 30 minutes after removing the adhesive. These lesions can be mechanical, such as skin stripping, blisters, or tears, or they can be dermatitis, such as allergic or irritant contact dermatitis. MARSI can also cause maceration and folliculitis.

MARSI can occur in any patient group because medical adhesive is used in most care settings. For example, adhesive is used in dressings for wound protection or management, as well as in those to stabilize cannulas for intravenous therapy or to position electrocardiograph dots. Patients at an increased risk of developing MARSI include: the older population with dermal fragility, infants in neonatal intensive care who may have an underdeveloped epidermis, patients with malnutrition and dehydration because normal/adequate hydration levels positively impact normal skin physiology, patients on medications such as corticosteroids or those requiring radiation treatment, which can induce abnormalities in lipid synthesis and the intercellular structure of the stratum corneum, in turn prolonging epidermal barrier recovery, patients with dermal pathologies such as eczema, an patients requiring frequent dressing changes.

The occurrence of MARSI is generally underreported. While the injuries may initially be considered minor, the care and management of these can be expensive. Moreover, the injuries over time can be quire debilitating. Depending upon the recovery can take from 1 to 8 weeks depending on the cause of MARSI, which may require a great deal of nursing time.

Barrier products and adhesive removers are currently available on the market; however, these products do not promote skin health if an injury has occurred due to the placement or removal of a medical device. Thus, there is a need for a product that can treat or prevent the onset of medical adhesive-related skin injury (MARSI) and other related complications associated with medical devices attached to patients.

### SUMMARY

In accordance with the purpose(s) of the present disclosure, as embodied and broadly described herein, the disclosure, in one aspect, relates to personal hygiene wipes designed to address the unique skin challenges faced by individuals. In one aspect, the personal hygiene wipes described herein can provide relief to a subject that is wearing or using medical devices and topically worn appliances. In another aspect, the use of the wipes described herein can treat or prevent the onset of medical adhesive-related skin injury (MARSI), which is a persistent and debilitating injury for a wide range of patient groups.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “an excipient” include, but are not limited to, mixtures or combinations of two or more such excipients, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value.

Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range. Thus, for example, if a component is in an amount of about 1%, 2%, 3%, 4%, or 5%, where any value can be a lower and upper endpoint of a range, then any range is contemplated between 1% and 5% (e.g., 1% to 3%, 2% to 4%, etc.).

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used interchangeably herein, “subject,” “individual,” or “patient” can refer to a vertebrate organism, such as a mammal (e.g., human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as, for example, eye disease. The effect can be therapeutic in terms of a partial or complete cure of a disease, condition, symptom or adverse effect attributed to the disease, disorder, or condition. The term “treatment” includes any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “therapeutic” can refer to treating, healing, and/or ameliorating a disease, disorder, condition, or side effect, or to decreasing in the rate of advancement of a disease, disorder, condition, or side effect.

As used herein, “effective amount” can refer to the amount of a disclosed compound or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “improves,” “improvement,” or “improving” refers to enhancing one or more physical properties or features of a subject after being administered the unit dose compositions described herein.

The term “pharmaceutically acceptable” describes a material that is not biologically or otherwise undesirable, i.e., without causing an unacceptable level of undesirable biological effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

As used herein, the term “wipe” means a type of fabric article suitable for cleansing, disinfecting, applying a compound, or removing a compound from a surface (e.g., the skin of a subject).

As used herein, the term “flushable” means the ability of a material, when flushed, to clear the toilet, drain lines, and screens leading to the municipal wastewater conveyance system.

As used herein, the term “dispersible” means the ability of a material to readily break apart in water. In particular, the term “dispersible” means the ability of a material to readily break apart due to the physical forces encountered during flushing in a common toilet, conveyance in a common wastewater system, and processing in a common treatment system.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e., one atmosphere).

### Personal Hygiene Wipes and Methods of Making and Using the Same

Described herein are personal hygiene wipes designed to address the unique skin challenges faced by individuals. The wipes described herein include components that provide several beneficial properties that enhance the skin of a subject when applied.

In one aspect, the wipes described herein are applied to the skin of a subject, where the subject is currently wearing a medical device. These individuals frequently experience skin irritation, and allergic reactions, due to the repeated application and removal of adhesives present on the medical devices. The wipes described alleviate these issues.

In one aspect, the wipes described herein can treat or prevent the onset of medical adhesive-related skin injury (MARSI), which is a persistent and debilitating injury for a wide range of patient groups. Medical adhesive-related skin injuries occur when superficial layers of skin are removed by medical adhesive. Medical adhesives are required for the fixation of numerous medical devices (e.g., intravenous injections, central lines, and various catheters and monitors, etc.). For example, subject's having diabetes typically use one or more topical medical devices such as, for example, insulin pumps and continuous glucose monitors. Peeling off adhesive dressings and devices removes loosely bound epidermal cells and stratum corneum, thereby compromising the skin barrier and increasing transepidermal water loss, which ultimately results in skin breakdown and medical adhesive-related skin injuries.

MARSI can occur in any patient group because medical adhesive is used in most care settings. For example, adhesive is used in dressings for wound protection or management, as well as in those to stabilize cannulas for intravenous therapy or to position electrocardiograph dots. Patients at an increased risk of developing MARSI include: the older population with dermal fragility, infants in neonatal intensive care who may have an underdeveloped epidermis, patients with malnutrition and dehydration because normal/adequate hydration levels positively impact normal skin physiology, patients with diabetes, patients on medications such as corticosteroids or those requiring radiation treatment, which can induce abnormalities in lipid synthesis and the intercellular structure of the stratum corneum, in turn prolonging epidermal barrier recovery, patients with dermal pathologies such as eczema, an patients requiring frequent dressing changes.

The use of the wipes described herein can be used to treat one or more pre-existing medical adhesive-related skin injuries. Examples of such injuries include, but are not limited to, irritant contact dermatitis, allergic dermatitis, maceration of the skin, folliculitis, blisters, and bulla. The injury can persist longer than 30 minutes after removal of the adhesive. MARSI not only affects skin integrity, but also causes pain, increases risk of infection, potentially increases wound size, and delays healing. The wipes described herein include several components as discussed below that can reduce or minimize the extent of the injuries and promote wound healing.

In addition to treating one or more pre-existing medical adhesive-related skin injuries, the wipes described herein can prevent the onset of medical adhesive-related skin injuries. The components present in the wipes described herein can promote skin health, which makes the skin of the subject less susceptible to MARSI.

In one aspect, the wipes described can applied to the surface of the skin of the subject prior to applying a medical device to the skin. In another aspect, the wipe is applied to the surface of the skin of the subject after removing a medical device from the skin. The wipe can be applied to the skin of the subject for a sufficient time such to ensure the components present on the are transferred from the wipe to the skin of the subject.

In one aspect, the wipes described herein include is a wet wipe that includes one or more components present in a fabric. In one aspect, the wipe includes a fabric substrate impregnated with a composition comprising a solvent, a corticosteroid, and a natural astringent. In one aspect, the fabric substrate is soaked or immersed in the composition composed of the solvent, the corticosteroid, and the natural astringent. In one aspect, the solvent is water, an alcohol (e.g., ethanol), or a combination thereof. After wipe has immersed in the composition, the wipe is ready for use. The wipe can be packaged as typically done with traditional wet wipes.

The wipes described herein include a corticosteroid. Corticosteroids are a class of steroid hormones that are produced in the adrenal cortex of vertebrates, as well as the synthetic analogues of these hormones. Corticosteroids can reduce inflammation and itching of the skin of the subject.

In one aspect, the corticosteroid is a synthetic or naturally occurring compound. In one aspect, the corticosteroid is a glucocorticoid. In another aspect, the corticosteroid is a mineralocorticoid. Examples of corticosteroids useful herein include, but are not limited to, fluticasone propionate, hydrocortisone, ethamethasoneb, prednisone, methylprednisolone, prednisolone, dexamethasone, triamcinolone, bethamethasone, or any combination thereof. In one aspect, the amount of corticosteroid present in the composition used to produce the wipes described herein is from about 25 mcg to about 200 mcg. In another aspect, the amount of corticosteroid present in the composition is about 25 mcg, 50 mcg, 75 mcg, 100 mcg, 125 mcg, 150 mcg, 175 mcg, or 200 mcg, where any value can be a lower and upper endpoint of a range (e.g., 50 mcg to 150 mcg).

The wipes described herein include a natural astringent. Astringents can reduce inflammation as well as help cleanse the skin to promote skin health. In one aspect, natural astringent includes alum, acacia, sage, yarrow, witch hazel, bayberry, distilled vinegar, rubbing alcohol, or any combination thereof.

In one aspect, the amount of natural astringent present in the composition used to produce the wipes described herein is from about 1.0% w/w to about 10.0% w/w of the composition. In another aspect, the amount of natural astringent present in the composition used to produce the wipes is about 1.0% w/w, 1.5% w/w, 2.0% w/w, 2.5% w/w, 3.0% w/w. 3.5% w/w, 4.0% w/w, 4.5% w/w, 5.0% w/w, 5.5% w/w, 6.0% w/w, 6.5% w/w, 7.0% w/w, 7.5% w/w, 8.0% w/w, 8.5% w/w, 9.0% w/w. 9.5% w/w, or 10.0% w/w of the composition, where any value can be a lower and upper endpoint of a range (e.g., 5.0% w/w to 7.5% w/W).

The compositions used to produce the wipes described herein can include one or more additional components to impart additional benefits to the subject. In one aspect, the compositions used to produce the wipes include one or more of an antihistamine, a skin healing agent, a soothing agent, an antimicrobial agent, or any combination thereof.

Antihistamines can reduce or prevent allergic reactions on the surface of the skin. In one aspect, the antihistamine includes diphenhydramine/zinc acetate, diphenhydramine, doxepin, azelastine, azelastine HCl, or any combination thereof. In one aspect, the amount of antihistamine present in the composition used to produce the wipes described herein is from about 0.05% w/w to about 0.5% w/w of the composition. In another aspect, the amount of natural astringent present in the composition used to produce the wipes about 0.05% w/w, 0.10% w/w, 0.15% w/w, 0.20% w/w, 0.25% w/w. 0.30% w/w, 0.35% w/w, 0.40% w/w, 0.45% w/w, or 0.50% w/w of the composition, where any value can be a lower and upper endpoint of a range (e.g., 0.15% w/w to 0.30% w/w).

Skin healing agents can promote skin healing and regeneration. In one aspect, the skin healing agent includes calendula oil, cucumber oil, lavender oil, olive oil, coconut oil, capryl caprylate triglyceride, shea butter, D-panthenol, glycerine, allantoin, oatmeal extract, and any combination thereof.

In one aspect, the amount of skin healing agent present in the composition used to produce the wipes described herein is from about 0.1% w/w to about 5.0% w/w of the composition. In another aspect, the amount of skin healing agent present in the composition used to produce the wipes is about 0.1, % w/w, 0.5% w/w, 1.0% w/w, 1.5% w/w, 2.0% w/w, 2.5% w/w, 3.0% w/w, 3.5% w/w, 4.0% w/w, 4.5% w/w, or 5.0% w/w of the composition, where any value can be a lower and upper endpoint of a range (e.g., 1.5% w/w to 3.5% w/w).

Soothing agents can help moisturize the skin and promote skin health. In one aspect, the soothing agent is aloe e.g., an aloe gel, aloe paste, aloe extract, aloe powder, or combinations thereof. Aloe vera gel is separated from the rind of the Aloe vera plant by filleting an aloe leaf, separating the inner gel from the outer leaf rind and separating it from the yellow sap contained within the rind. The inner gel is then homogenized and available for use or further processing either as a concentrate by removal of water or more extensive processing to create an extract. The Aloe vera gel contains a variety of chemical substances with a large molecular weight complex carbohydrate, identified and given the United States Adopted Name (USAN), acemannan.

In one aspect, the amount of soothing agent present in the composition used to produce the wipes described herein is from about 1.0% w/w to about 20.0% w/w of the composition. In another aspect, the amount of natural astringent present in the composition used to produce the wipes is about 1.0% w/w, 2.0% w/w, 3.0% w/w, 4.0% w/w, 5.0% w/w, 6.0% w/w, 7.0% w/W, 8.0% w/w, 9.0% w/w, 10.0% w/w, 11.0% w/w, 12.0% w/w, 13.0% w/w, 14.0% w/w, 15.0% w/w, 16.0% w/w, 17.0% w/w, 18.0% w/w, 19.0% w/w, or 20.0% w/w of the composition, where any value can be a lower and upper endpoint of a range (e.g., 5.0% w/w to 15.0% w/w).

Antimicrobial agents can promote skin health by reducing the likelihood of skin infection. In one aspect, the antibacterial agent includes chlorohexidine, gentian violet, hydrogen peroxide, povidone-iodine, triclosan, hypochlorous acid, or any combination thereof.

In one aspect, the amount of antimicrobial agent present in the composition used to produce the wipes described herein is from about 0.01% w/w to about 0.5% w/w of the composition. In another aspect, the amount of antimicrobial agent present in the composition used to produce the wipes about 0.01% w/w, 0.05% w/w, 0.10% w/w, 0.15% w/w, 0.20% w/w, 0.25% w/w, 0.30% w/w, 0.35% w/w, 0.40% w/w, 0.45% w/w, or 0.50% w/w of the composition, where any value can be a lower and upper endpoint of a range (e.g., 0.15% w/w to 0.30% w/w).

Additional components can be used incorporated into the wipes described herein, including but not limited to, benzethonium chloride, ethanol, hydrastis canadensis L., hydrogen peroxide, panax quinquefolis L., sodium chloride, sodium hypochlorite, berberine, a phosphate, or any combination thereof.

In one aspect, the compositions and wipes described herein include fluticasone propionate (corticosteroid) and witch hazel (natural astringent), which provide synergistic properties. Fluticasone propionate is a potent corticosteroid, reducing inflammation by suppressing the immune response. Witch hazel also possesses anti-inflammatory properties due to its tannins and flavonoids. Together, they offer a dual-action approach to calming irritated skin. Furthermore, fluticasone propionate helps to repair the skin barrier, preventing further irritation. Witch hazel's astringent properties tighten the skin, reducing its permeability. Thus, the combination of fluticasone propionate and witch hazel can improve the skin's barrier function and properties.

In another aspect, when an antihistamine is used, there can be further synergy. For example, fluticasone propionate tackles inflammation, a key component of allergic reactions. Azelastine HCl, as an antihistamine, blocks histamine, the chemical responsible for allergy symptoms like itching and redness. Their combined action provides comprehensive relief from both the cause and symptoms of allergic reactions.

In another aspect, when a soothing agent is used, there can be further synergy. For example, the natural astringent (e.g., witch hazel) can help to tighten and tone the skin, while aloe vera's polysaccharides and glycoproteins deeply hydrate and soothe. This combination is particularly beneficial for skin that is irritated and dry from adhesive use.

The compositions used to produce the wipes described herein can be produced by mixing the components described herein in a solvent. In one aspect, the solvent is water, an alcohol, or a combination there of. In one aspect, solvent is electrolyzed water. Electrolyzed water is produced by the electrolysis of ordinary tap water containing dissolved sodium chloride. The electrolysis of such salt solutions produces a solution of hypochlorous acid and sodium hydroxide.

In one aspect, the amount of solvent present in the composition used to produce the wipes described herein is from about 1.0% w/w to about 50.0% w/w of the composition. In another aspect, the amount of natural astringent present in the composition used to produce the wipes is about 1.0% w/w, 5.0% w/w, 10.0% w/w, 15.0% w/w, 20.0% w/w, 25.0% w/w, 30.0% w/w, 35.0% w/w, 40.0% w/w, 45.0% w/w, or 50.0% w/w of the composition, where any value can be a lower and upper endpoint of a range (e.g., 5.0% w/w to 15.0% w/w).

The pH of the composition can also be modified as needed. In one aspect, the composition used to produce the wipes described herein has a pH of about 4.5 to about 6.5. In another aspect, the composition used to produce the wipes described herein has a pH of about 4.5, 5.0, 5.5, 6.0, or 6.5, where any value can be a lower and upper endpoint of a range (e.g., 5.0 to 6.0).

The fabric substrate used to produce the wipes described herein can be prepared from any suitable material typically used to prepare wipes applied to the skin of a subject. In one aspect, the fabric substrate can include a nonwoven material. In one aspect, the nonwoven material can include natural or cellulose-based fibers, such as those derived from wood pulp. synthetic cellulose-based fibers, or any mixture thereof. Examples of cellulose fibers include, but are not limited to, digested fibers, such as kraft fibers, kraft digested fibers, pre-hydrolyzed kraft fibers, pre-hydrolyzed kraft digested fibers, soda fibers, sulfite fibers, and chemi-thermal mechanical and thermo-mechanical treated fibers derived from softwood, hardwood or cotton linters. Non-limiting examples of cellulose fibers suitable for use in this invention include the cellulose fibers derived from softwoods, such as pines, firs, and spruces.

In another aspect, the fabric substrate may be a woven material. In one aspect, fabric substrate can be a woven structure that utilizes monofilaments (yarns, threads) made of synthetic polymers (usually polyethylene, polypropylene, or nylon) that may be overlaid with a patterned polymer resin. The structured fabric may be produced using any of various processes for making a three-dimensional object primarily through additive processes in which successive layers of material are laid down under computer control.

In one aspect, the fabric substrate is water dispersible. In this aspect, the fabric substrate substantially disperses into smaller pieces and individual fibers in a relatively short time under immersion and mild agitation in water, such as being present in a standard septic tank or sanitary system. The ability to biodegrade and disperse in a septic tank or sanitary system prevents the wipe from clogging up sewage lines and screens at sewage treatment plants. Thus, the water dispersible substrate can be flushed down a toilet after a single use.

Now having described the aspects of the present disclosure, in general, the following Aspects describe some additional embodiment of the present disclosure.

Aspect 1. A personal hygiene wipe comprising a fabric substrate and the following components:
(a) a corticosteroid,
(b) a natural astringent, and
(c) a solvent

Aspect 2. The wipe of Aspect 1, wherein the corticosteroid comprises fluticasone propionate, hydrocortisone, ethamethasoneb, prednisone, methylprednisolone, prednisolone, dexamethasone, triamcinolone, bethamethasone, or any combination thereof.

Aspect 3. The wipe of Aspect 1 or 2, wherein the natural astringent comprises are alum, acacia, sage, yarrow, witch hazel, bayberry, distilled vinegar, rubbing alcohol, or any combination thereof.

Aspect 4. The wipe of any one of Aspects 1-3, wherein the wipe further comprises an antihistamine.

Aspect 5. The wipe of Aspect 4, wherein the antihistamine comprises diphenhydramine/zinc acetate, diphenhydramine, doxepin, azelastine, azelastine HCl, or any combination thereof.

Aspect 6. The wipe of any one of Aspects 1-5, wherein the solvent comprises water, an alcohol, or a combination thereof.

Aspect 7. The wipe of any one of Aspects 1-5, wherein the solvent comprises electrolyzed water.

Aspect 8. The wipe of any one of Aspects 1-7, wherein the wipe further comprises a skin healing agent.

Aspect 9. The wipe of Aspect 8, wherein the skin healing agent comprises calendula oil, cucumber oil, lavender oil, olive oil, coconut oil, capryl caprylate triglyceride, shea butter, D-panthenol, glycerine, allantoin, oatmeal extract, and any combination thereof.

Aspect 10. The wipe of any one of Aspects 1-9, wherein the wipe further comprises a soothing agent.

Aspect 11. The wipe of Aspect 10, wherein soothing agent comprises aloe.

Aspect 12. The wipe of any one of Aspects 11, wherein the wipe further comprises an antibacterial agent.

Aspect 13. The wipe of any one of Aspects 1-12, wherein the antibacterial agent comprises an chlorohexidine, gentian violet, hydrogen peroxide, povidone-iodine, triclosan, hypochlorous acid, or any combination thereof.

Aspect 14. The wipe of Aspect 1, wherein the wipe comprises the following components:
(a) a corticosteroid,
(b) an antihistamine,
(c) a natural astringent,
(d) a skin healing agent,
(e) a skin moisturizer,
(f) an antimicrobial agent, and
(e) a solvent.

Aspect 15. The wipe of Aspect 1, wherein the wipe comprises the following components:
(a) fluticasone propionate
(b) azelastine HCl
(c) witch hazel extract
(d) allantoin
(e) aloe vera gel,
(f) hypochlorous acid, and
(g) electrolyzed water.

Aspect 16. The wipe of any one of Aspects 1-15, wherein the wipe comprises a fabric substrate impregnated with a composition comprising a solvent, a corticosteroid and the natural astringent, and optionally one or more of an antihistamine, a skin healing agent, a soothing agent, an antimicrobial agent, or any combination thereof.

Aspect 17. The wipe of Aspect 16, wherein the corticosteroid is from about 25 mcg to about 200 mcg in the composition.

Aspect 18. The wipe Aspect 16 or 17, wherein the natural astringent is from about 1.0% w/w to about 10.0% w/w of the composition.

Aspect 19. The wipe of any one of Aspects 16-18, wherein when the antihistamine is present, the antihistamine is from about 0.05% w/w to about 0.5% w/w of the composition.

Aspect 20. The wipe of any one of Aspects 16-19, wherein when the skin healing agent is present, the skin healing agent is from about 0.1% w/w to about 5.0% w/w of the composition.

Aspect 21. The wipe of any one of Aspects 16-20, wherein when the soothing agent is present, the soothing agent is from about 1.0% w/w to about 20.0% w/w of the composition.

Aspect 22. The wipe of any one of Aspects 16-21, wherein when the antimicrobial agent is present, the antimicrobial agent is from about 0.01% w/w to about 0.5% w/w of the composition.

Aspect 23. The wipe of any one of Aspects 16-22, wherein the solvent comprise water, an alcohol or a combination thereof.

Aspect 24. The wipe of any one of Aspects 16-22, wherein the solvent comprise electrolyzed water.

Aspect 25. The wipe of any one of Aspects 16-24, wherein the solvent is from about 1.0% w/w to about 50.0% w/w of the composition.

Aspect 26. The wipe of Aspect 16, wherein the composition comprises
(a) fluticasone propionate in the amount of about 25 mcg to about 200 mcg in the composition;
(b) azelastine HCl in the amount of about 0.05% w/w to about 0.5% w/w of the composition;
(c) witch hazel extract in the amount of about 1.0% w/w to about 10.0% w/w of the composition;
(d) allantoin in the amount of about 0.1% w/w to about 5.0% w/w of the composition;
(e) aloe vera gel in the amount of about 1.0% w/w to about 20.0% w/w of the composition;
(f) hypochlorous acid in the amount of about 0.01% w/w to about 0.5% w/w of the composition; and
(g) electrolyzed water in the amount of about 1.0% w/w to about 50.0% w/w of the composition.

Aspect 27. The wipe of any one of Aspects 16-26, wherein the composition further comprises benzethonium chloride, ethanol, hydrastis canadensis L., hydrogen peroxide, panax quinquefolis L., sodium chloride, sodium hypochlorite, berberine, a phosphate, or any combination thereof.

Aspect 28. The wipe of any one of Aspects 16-27, wherein the composition has a pH of about 4.5 to about 6.5.

Aspect 29. The wipe of any one of Aspects 1-28, wherein the fabric substrate comprises a non-woven fabric.

Aspect 30. The wipe of any one of Aspects 1-28, wherein the fabric substrate comprises natural or cellulose based fibers.

Aspect 31. The wipe of any one of Aspects 1-30, wherein the fabric substrate comprises a water dispersible material.

Aspect 32. The wipe of any one of Aspects 1-31, wherein the fabric substrate is textured.

Aspect 33. The wipe of any one of Aspects 1-32, wherein the wipe is a baby wipe or a cosmetic wipe.

Aspect 34. A method for using the wipe of any one of Aspects 1-33, wherein the method comprises applying the wipe to the surface of the skin of a subject.

Aspect 35. The method of Aspect 34, wherein the wipe is applied to the surface of the skin of the subject prior to applying a medical device to the skin.

Aspect 36. The method of Aspect 34, wherein the wipe is applied to the surface of the skin of the subject after removing a medical device from the skin.

Aspect 37. A method for treating a medical adhesive-related skin injury (MARSI), wherein the method comprises applying the wipe of any one of Aspects 1-33 to the injury on the subject.

Aspect 38. A method for preventing the onset of a medical adhesive-related skin injury (MARSI), wherein the method comprises applying the wipe of any one of Aspects 1-33 to the skin of a subject prior to and/or after the application of a medical device with an adhesive to the skin of the subject.

Aspect 39. The method of Aspect 37 or 38, wherein the patient is an elderly patient, an infants in neonatal intensive care who may have an underdeveloped epidermis, a patient with malnutrition and dehydration because normal/adequate hydration levels positively impact normal skin physiology, a patient with diabetes, a patients on medication such as a corticosteroid or those requiring radiation treatment, a patient with dermal pathologies such as eczema, or a patient requiring frequent dressing changes.

Aspect 40. The method of any one of Aspects 37-39, wherein the injury comprises irritant contact dermatitis, allergic dermatitis, maceration of the skin, folliculitis, blisters, bulla, and any combination thereof.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

1. A personal hygiene wipe comprising a fabric substrate and the following components:
(a) a corticosteroid,
(b) a natural astringent, and
(c) a solvent

2. The wipe of claim 1, wherein the corticosteroid comprises fluticasone propionate, hydrocortisone, ethamethasoneb, prednisone, methylprednisolone, prednisolone, dexamethasone, triamcinolone, bethamethasone, or any combination thereof.

3. The wipe of claim 1, wherein the natural astringent comprises are alum, acacia, sage, yarrow, witch hazel, bayberry, distilled vinegar, rubbing alcohol, or any combination thereof.

4. The wipe of claim 1, wherein the wipe further comprises an antihistamine selected from the group consisting of diphenhydramine/zinc acetate, diphenhydramine, doxepin, azelastine, azelastine HCl, and any combination thereof.

5. The wipe of claim 1, wherein the solvent comprises water, an alcohol, or a combination thereof.

6. The wipe of claim 1, wherein the solvent comprises electrolyzed water.

7. The wipe of claim 1, wherein the wipe further comprises a skin healing agent selected from the group consisting of calendula oil, cucumber oil, lavender oil, olive oil, coconut oil, capryl caprylate triglyceride, shea butter, D-panthenol, glycerine, allantoin, oatmeal extract, and any combination thereof.

8. The wipe of claim 1, wherein the wipe further comprises a soothing agent.

9. The wipe of claim 8, wherein soothing agent comprises aloe.

10. The wipe of claim 1, wherein the wipe further comprises an antibacterial agent selected from the group consisting of chlorohexidine, gentian violet, hydrogen peroxide, povidone-iodine, triclosan, hypochlorous acid, and any combination thereof.

11. The wipe of claim 1, wherein the wipe comprises the following components:
(a) a corticosteroid,
(b) an antihistamine,
(c) a natural astringent,
(d) a skin healing agent,
(e) a skin moisturizer,
(f) an antimicrobial agent, and
(e) a solvent.

12. The wipe of claim 1, wherein the wipe comprises the following components:
(a) fluticasone propionate
(b) azelastine HCl
(c) witch hazel extract
(d) allantoin
(e) aloe vera gel,
(f) hypochlorous acid, and
(g) electrolyzed water.

13. The wipe of claim 1, wherein the wipe comprises a fabric substrate impregnated with a composition comprising a solvent, a corticosteroid and the natural astringent, and optionally one or more of an antihistamine, a skin healing agent, a soothing agent, an antimicrobial agent, or any combination thereof.

14. The wipe of claim 1, wherein the corticosteroid is from about 25 mcg to about 200 mcg in the composition.

15. The wipe of claim 1, wherein the natural astringent is from about 1.0% w/w to about 10.0% w/w of the composition.

16. The wipe of claim 1, wherein the composition comprises
(a) fluticasone propionate in the amount of about 25 mcg to about 200 mcg in the composition;
(b) azelastine HCl in the amount of about 0.05% w/w to about 0.5% w/w of the composition;
(c) witch hazel extract in the amount of about 1.0% w/w to about 10.0% w/w of the composition;
(d) allantoin in the amount of about 0.1% w/w to about 5.0% w/w of the composition;
(e) aloe vera gel in the amount of about 1.0% w/w to about 20.0% w/w of the composition;
(f) hypochlorous acid in the amount of about 0.01% w/w to about 0.5% w/w of the composition; and
(g) electrolyzed water in the amount of about 1.0% w/w to about 50.0% w/w of the composition.

17. The wipe of claim 1, wherein the wipe is a baby wipe or a cosmetic wipe.

18. A method for using the wipe of claim 1, wherein the method comprises applying the wipe to the surface of the skin of a subject.

19. A method for treating a medical adhesive-related skin injury (MARSI), wherein the method comprises applying the wipe oof claim 1 to the injury on the subject.

20. A method for preventing the onset of a medical adhesive-related skin injury (MARSI), wherein the method comprises applying the wipe of claim 1 to the skin of a subject prior to and/or after the application of a medical device with an adhesive to the skin of the subject.
